(19) United States
(12) Patent Application Publication
(10) Pub. No.: US 2005/0261342 A1
(43) Pub. Date: Nov. 24, 2005

(54) Metabolites of (+)-(2S,3S)-3(2-methoxy-5- trifluoromethoxybenzylamino)-2-phenyl-piperidine

(75) Inventors: Amin Mohamed Kamel, Mystic, CT (US); Kevin Albert Colizza, Mystic, CT (US); John A. Davis, Howell, MI (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/131,733

(22) Filed: May 17, 2005

Related U.S. Application Data
(60) Provisional application No. 60/573,555, filed on May 21, 2004.

(51) Int. Cl. A61K 31/445; C07D 207/10; C07D 211/92; A61K 31/40

(52) U.S. Cl. 514/317; 514/426; 546/223; 548/557

(57) ABSTRACT

Metabolites of (+)-(2S, 3S)-3-(2-methoxy-5-trifluoromethoxybenzylamino)-2-phenyl-piperidine, and use of same.

### FIELD OF THE INVENTION

The invention relates to compounds that are mammalian metabolites of (+)-(2S, 3S)-3-(2-methoxy-5-trifluoromethoxybenzylamino)-2-phenyl-piperidine, pharmaceutical compositions comprising such metabolites and the use of such metabolites in the treatment and prevention of inflammatory and central nervous system disorders, as well as several other disorders. The pharmaceutical metabolites of this invention are substance P receptor antagonists. This invention also relates to novel intermediates used in the synthesis of such substance P receptor antagonists.

### BACKGROUND OF THE INVENTION

Substance P is a naturally occurring undecapeptide belonging to the tachykinin family of peptides, the latter being named because of its prompt stimulatory action on smooth muscle tissue. More specifically, substance P is a pharmacologically active neuropeptide that is produced in mammals (having originally been isolated from gut) and possesses a characteristic amino acid sequence that is illustrated by D. F. Veber et al. in U.S. Pat. No. 4,680,283. The wide involvement of substance P and other tachykinins in the pathophysiology of numerous diseases has been amply demonstrated in the art. For instance, substance P has recently been shown to be involved in the transmission of pain or migraine (see B. E. B. Sandberg et al., Journal of Medicinal Chemistry, 25, 1009 (1982)), as well as in central nervous system disorders such as anxiety and schizophrenia, in respiratory and inflammatory diseases such as asthma and rheumatoid arthritis, respectively, in rheumatic diseases such as fibrositis, and in gastrointestinal disorders and diseases of the GI tract such as ulcerative colitis and Crohn's disease, etc. (see D. Regoli in “Trends in Cluster Headache,” edited by F. Sicuteri et al., Elsevier Scientific Publishers, Amsterdam, pp. 85-95 (1987)).

In the recent past, some attempts have been made to provide antagonists for substance P and other tachykinin peptides in order to more effectively treat the various disorders and diseases listed above. The few such antagonists thus far described are generally peptide-like in nature and are therefore too labile from a metabolic point of view to serve as practical therapeutic agents in the treatment of disease. The non-peptidic antagonists of the present invention, on the other hand, do not possess this drawback, being far more stable from a metabolic point of view than the agents referred to above.

Quinuclidine derivatives and related compounds that exhibit activity as substance P receptor antagonists. Piperidine derivatives and related heterocyclic nitrogen containing compounds that are useful as substance P antagonists.

The aforementioned patents and patent applications are all incorporated by reference in their entireties herein.

### SUMMARY OF THE INVENTION

In one practice, the invention relates to an isolated and purified metabolite of (+)-(2S, 3S)-3-(2-methoxy-5-trifluoromethoxybenzylamino)-2-phenyl-piperidine, or an analogue thereof, the racemic-diastereomeric mixtures and optical isomers thereof, the prodrugs thereof, and the pharmaceutically acceptable salts or solvates thereof.

In another practice, the invention relates to a pharmaceutical composition comprising an isolated and purified metabolite of (+)-(2S, 3S)-3-(2-methoxy-5-trifluoromethoxybenzylamino)-2-phenyl-piperidine having Formula I, or an analogue thereof, a racemic-diastereomeric mixture or optical isomer thereof, a prodrug thereof, or pharmaceutically acceptable salt or solvate thereof.

In another practice, the invention relates to methods of treating disease states associated with an excess of substance P using an isolated and purified metabolite of (+)-(2S, 3S)-3-(2-methoxy-5-trifluoromethoxybenzylamino)-2-phenyl-piperidine having Formula I, or an analogue thereof, or a pharmaceutical composition thereof, the racemic-diastereomeric mixtures and optical isomers thereof, the prodrugs thereof, and the pharmaceutically acceptable salts or solvates thereof.

### DETAILED DESCRIPTION OF THE INVENTION

In one practice, and without limitation, the invention relates to an isolated and purified metabolite of (+)-(2S, 3S)-3-(2-methoxy-5-trifluoromethoxybenzylamino)-2-phenyl-piperidine having Formula (I): 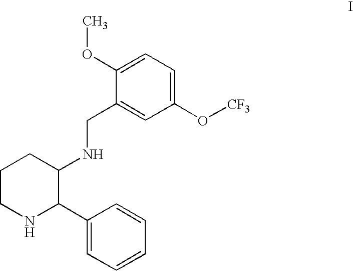
and analogues thereof, and pharmaceutically acceptable salts and solvates thereof.

The compounds of the invention are defined as metabolites of Formula I, or analogues thereof, or pharmaceutically acceptable salts or solvates thereof. The compounds of the invention include racemic-diastereomeric mixtures or optical isomers thereof, or prodrugs thereof. Preferred compounds of the invention are compounds of Formula II, III or IV hereinbelow, or pharmaceutically acceptable salts or solvates thereof.

Methods of making (+)-(2S, 3S)-3-(2-methoxy-5-trifluoromethoxybenzylamino)-2-phenyl-piperidine are disclosed in U.S. Pat. Nos. 5,744,480 and 5,733,450, the contents of which are incorporated herein by reference.

Unless otherwise indicated:

“Halogen” and “halo” and the like includes fluoro, chloro, bromo and iodo.

“Alkyl” including as appears in any terms such as “alkoxy” and “alkyoxycarbonyl,” or in any substutuents such as —O-(C₁-C₆)alkyl, —O-(C₁-C₆)alkyl, or -(C₁-C₆)alkyl-C(O)—R⁶ includes saturated monovalent hydrocarbon radicals having straight or branched moieties. Examples of alkyl groups include, but are not limited to, methyl, ethyl, n-propyl, isopropyl, and t-butyl.

“Cycloalkyl” includes non-aromatic saturated cyclic alkyl moieties wherein alkyl is as defined above. The cycloalkyl can be optionally substituted with one or more “ring system substituents” which can be the same or different, and are as defined above. Examples of cycloalkyl include, but are not limited to, cyclopropyl, cyclobutyl, cyclopentyl, cyclohexyl, and cycloheptyl; and bicycloalkyl and tricycloalkyl groups that are non-aromatic saturated carbocyclic groups consisting of two or three rings respectively, wherein said rings share at least one carbon atom. For purposes of the present invention, and unless otherwise indicated, bicycloalkyl groups include spiro groups and fused ring groups. Examples of bicycloalkyl groups include, but are not limited to, bicyclo-[3.1.0]-hexyl, bicyclo-2.2.1]-hept-1-yl, norbornyl, spiro[4.5]decyl, spiro[4.4]nonyl, spiro[4.3]octyl, and spiro[4.2]heptyl. An example of a tricycloalkyl group is adamantanyl. Cycloalkyl groups also include groups that are substituted with one or more oxo moieties. Examples of such groups with oxo moieties are oxocyclopentyl and oxocyclohexyl.

“Aryl” refers to monocyclic and multicyclic groups which includes an organic radical derived from an aromatic hydrocarbon by removal of one hydrogen, such as phenyl, naphthyl, tetrahydonaphthyhl, indenyl, indanyl, and fluorenyl; and fused ring groups wherein at least one ring is aromatic. The aryl groups can be optionally substituted with one or more “ring system substituents” which can be the same or different, and are as defined above. The aryl groups of this invention can also include ring systems substituted with one or more oxo moieties.

“Alkoxy” refers to an —O-alkyl group wherein alkyl is defined above.

“Oxo” is ═O.

“Heterocycloalkyl” refers to non-aromatic cyclic groups containing one or more heteroatoms, preferably from one to four heteroatoms, each selected from O, S and N. The heterocycloalkyl can be optionally substituted with one or more “ring system. The term “one or more substituents,” as used herein, includes from one to the maximum number of substituents possible based on the number of available bonding sites.

“Treating” “treatment: and like terms refer to reversing, alleviating, or inhibiting the progress of a disorder or condition. As used herein, “treatment: and “treating” and like terms can also refer to decreasing the probability or incidence or occurrence of a disease or condition in a mammal compared to an untreated control population, or in the same mammal prior to treatment, according to the present invention. “Treatment” or “treating” can also include delaying or preventing the onset of a disease or condition. “Treatment” or “treating” as used herein also encompasses preventing the recurrence of disease or condition. “Treatment” or “treatment” a disorder or condition also encompasses treating (as used herein) on ore more symptoms of the disorder or condition.

“Gluc.” refers to a glucoronide substituent. Glucuronic acid reacts with an acid or alcohol or phenol moiety on the metabolite or parent compound to form the “glucuride.” Glucuronic acid is the substituent that is transferred to a metabolite from the phase II conjunction reaction of glucuronidation.

“Subject” is an animal, including mammals, and including human beings.

“Mammal” refers to any member of the class “Mammalia”, including, but not limited to, humans, dogs, and cats.

“Isolated and purified” includes susbtantially pure and isolated sufficient for purposes of the invention as understood by the artisan.

The invention includes isotopically-labeled metabolites identical to those of Formulae (I) to (XXIV) and other metabolites of the invention save for one or more atoms being replaced by one of atomic mass or mass number different from that usually found in nature as understood by the artisan.

“Co-administration” of a combination of a (+)-(2S, 3S)-3-(2-methoxy-5-trifluoromethoxybenzylamino)-2-phenyl-piperidine metabolite and an additional compound or additional compounds means that these components can be administered together as a composition or as part of the same, unitary dosage form. “Co-administration” also includes administering a (+)-(2S, 3S)-3-(2-methoxy-5-trifluoromethoxybenzylamino)-2-phenyl-piperidine metabolite and an additional compound or additional compounds separately but as part of the same therapeutic treatment program or regimen. The components need not necessarily be administered at essentially the same time, although they can if so desired. Thus “co-administration” includes, for example, administering a (+)-(2S, 3S)-3-(2-methoxy-5-trifluoromethoxybenzylamino)-2-phenyl-piperidine metabolite and an additional compound as separate dosages or dosage forms, but at the same time. “Co-administration” also includes separate administration at different times and in any order. For example, where appropriate a patient can take one or more component(s) of the treatment in the morning and the one or more of the other component(s) at night.

“Solvates” of the compounds of the invention are also contemplated herein. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂.

The term “prodrug” means compounds that are transformed in vivo to yield a metabolite of the present invention. The transformation can occur by various mechanisms, such as through hydrolysis in blood. A good discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a metabolite of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-((C₁-C₂))alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₁-C₃)alkyl.

Similarly, if a metabolites of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α.-amino(C₁-C₄)alkanoyl, arylacyl and α-aminoacyl, or α-aminoacylα-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound of the present invention comprises an amine functional group, a prodrug can be formed by the replacement of a hydrogen atom in the amine group with a group such as R^{x}-carbonyl, R^{x}O-carbonyl, NR^{x} R^{x1}-carbonyl where R^{x} and R^{x1} are each independently ((C₁-C₁₀)alkyl, (C₃-C₇)cycloalkyl, benzyl, or R^{x}-carbonyl is a natural α-aminoacyl or natural α-aminoacyl-natural α-aminoacyl, —C(OH)C(O)OY^{x} wherein (Y^{x} is H, (C₁-C₆)alkyl or benzyl), —C(OY^{x0}) Y^{x} wherein Y^{x0} is (C₁-C₄)alkyl and Y^{x1} is ((C₁-C₆)alkyl, carboxy(C₁-C₆)alkyl, amino(C₁-C₄)alkyl or mono-N- or di-N,N-(C₁-C₆)alkylaminoalkyl, —C(Y^{x2})Y^{x3} wherein Y^{x2} is H or methyl and Y^{x3} is mono-N- or di-N,N-(C₁-C₆)alkylamino, morpholino, piperidin-1-yl or pyrrolidin-1-yl.

As used herein, the term “effective amount” means an amount of one or more compounds of the methods of the present invention that are capable of treating the specific diseases and pathological conditions. The specific dose of a compound administered according to this invention will, of course, be determined by the particular circumstances surrounding the case including, for example, the compound administered, the route of administration, the state of being of the subject, and the severity of the pathological condition being treated.

“Chemical dependency,” as used herein, means an abnormal craving or desire for, or an addiction to a drug. Such drugs are generally administered to the affected individual by any of a variety of means of administration, including oral, parenteral, nasal or by inhalation. Examples of chemical dependencies treatable by the methods of the present invention are dependencies on alcohol, nicotine, cocaine, heroin, phenobarbital, and benzodiazepines (e.g., Valium (trademark)). “Treating a chemical dependency,” as used herein, means reducing or alleviating such dependency.

In another practice, the invention relates to an isolated and purified compound having Formula (II): 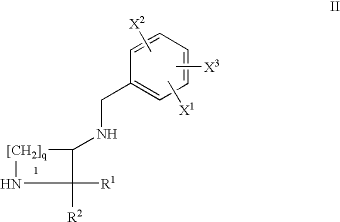
wherein X¹, X² and X³, which can be the same or different, are each independently selected from O-Gluc, —C(═O)OH, OH, —OSO₂OH, (C₁-C₁₀)alkoxy and (C₁-C₁₀)alkyl, wherein said (C₁-C₁₀)alkoxy and (C₁-C₁₀)alkyl are each optionally substituted with one to three halo atoms;

R¹ is a radical selected from the group consisting of H, (C₁-C₆) straight or branched alkyl, (C₃-C₇)cycloalkyl, (C₃-C₇)heterocycloalkyl, aryl, benzhydryl, and O-Gluc, wherein one of the phenyl moieties of said benzhydryl can optionally be replaced by naphthyl, and phenyl(C₁-C₆)alkyl-, wherein said aryl group and the phenyl moieties of said phenyl(C₁-C₆)alkyl- and benzhydryl can optionally be substituted with one or more substituents independently selected from halo, nitro, O-gluc, (C₁-C₁₀)alkyl optionally substituted with one to three halo atoms, (C₁-C₁₀)alkoxy optionally substituted with one to three halo atoms, amino, hydroxy-(C₁-C₆)alkyl, (C₁-C₆)alkoxy-(C₁-C₆)alkyl, (C₁-C₆)-alkylamino, (C₁-C₆)alkyl-O—C(═O)—, (C₁-C₆)alkyl-O—C(═O)-(C₁-C₆)alkyl, (C₁-C₆)alkyl-C(═O)—O—, (C₁-C₆)alkyl-C(═O)-(C₁-C₆)alkyl-O, (C₁-C₆)alkyl-C(═O)—, (C₁-C₆)alkyl-C(═O)-(C₁-C₆)alkyl-di-(C₁-C₆)alkylamino, —C(═O)NH-(C₁-C₆)alkyl, (C₁-C₆)-alkyl-C(═O)—NH-(C₁-C₆)alkyl, —NHC(═O)H and —NHC(═O)-(C₁-C₆)alkyl; and;

R² is hydrogen, phenyl or (C₁-C₆)alkyl; or

R¹ and R², together with the carbon to which they are attached, form a saturated carbocyclic ring having 3 to 7 carbon atoms wherein one of said carbon atoms can optionally be replaced by oxygen, nitrogen or sulfur;

q is an integer from 1-5;

ring 1 can be substituted with 1 or 2 oxo groups;

and pharmaceutically acceptable salts and solvates thereof.

with the provisos that when X¹ is —OCH₃, X² is not —OCF₃, and when X² is —OCH₃, X¹ is not —OCF₃.

In a preferred embodiment of this practice, X¹ and X², which can be the same or different, are each (C₁-C₃)alkoxy optionally substituted with one to three fluorine atoms; R¹ is aryl, R² is H, and q is 2.

In another preferred embodiment of this practice, X¹ and X², which can be the same or different, are each —OCF₃, R¹ is phenyl, R² is H and q is 2.

In another practice, the invention relates to an isolated and purified compound having Formula III: 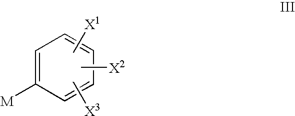
wherein X¹, X² and X³, which can be the same or different, are independently selected from the group consisting of halo, hydrogen, nitro, O-Gluc, (C₁-C₁₀)alkyl optionally substituted with one to three halo atoms, (C₁-C₁₀)alkoxy optionally substituted with one to three halo atoms,

—O—SO₂—OH, trifluoromethyl, hydroxy, phenyl, cyano, amino, (C₁-C₆)-alkylamino, di-(C₁-C₆)alkylamino, —C(═O)—NH-(C₁-C₆)alkyl, (C₁-C₆)alkyl-C(═O)—NH-(C₁-C₆)alkyl, hydroxy(C₁-C₄)alkyl, (C₁-C₄)alkoxy(C₁-C₄)alkyl, —NHC(═O)H and —NHC(═O)-(C₁-C₆)alkyl;

M is selected from the group consisting of —C(═O)—R³, —C(═O)—O—R³, -(C₁-C₆)alkyl-C(═O)—R³, -(C₁-C₆)alkyl-C(═O)—OR⁴, and -(C₁-C₆)alkyl-NR⁵R⁶;

R³ and R⁴, which can be the same or different, are each independently selected from the group consisting of H, (C₁-C₆) straight or branched alkyl, (C₃-C₇)cycloalkyl, (C₃-C₇)heterocycloalkyl, aryl, (C₁-C₆)aryl, benzhydryl wherein one of the phenyl moieties of said benzhydryl can optionally be replaced by naphthyl, and wherein said aryl group and aryl moiety of said aryl(C₁-C₆)alkyl- and benzhydryl can optionally be substituted with one or more substituents independently selected from the group consisting of halo, nitro, (C₁-C₁₀)alkyl optionally substituted with one to three halo atoms, (C₁-C₁₀)alkoxy optionally substituted with one to three halo atoms, amino, hydroxy-(C₁-C₆)alkyl, (C₁-C₆)alkoxy-(C₁-C₆)alkyl, (C₁-C₆)-alkylamino, (C₁-C₆)alkyl-O—C(═O)—, (C₁-C₆)alkyl-O—C(═O)-(C₁-C₆)alkyl, (C₁-C₆)alkyl-C(═O)—O—, (C₁-C₆)alkyl-C(═O)-(C₁-C₆)alkyl-O, (C₁-C₆)alkyl-C(═O)—, (C₁-C₆)alkyl-C(═O)-(C₁-C₆)alkyl-di-(C₁-C₆)alkylamino, —C(═O)NH-(C₁-C₆)alkyl, (C₁-C₆)-alkyl-C(═O)—NH-(C₁-C₆)alkyl, —NHC(═O)H and —NHC(═O)-(C₁-C₆)alkyl;

R⁵ and R⁶, which can be the same or different, are each independently selected from the group consisting of H, (C₁-C₆) straight or branched alkyl, (C₃-C₇)cycloalkyl, (C₃-C₇)heterocycloalkyl, aryl, (C₁-C₆)aryl, benzhydryl wherein one of the phenyl moieties of said benzhydryl can optionally be replaced by naphthyl, and wherein said aryl group and aryl moiety of said aryl(C₁-C₆)alkyl- and benzhydryl can optionally be substituted with one or more substituents independently selected from the group consisting of halo, nitro, (C₁-C₁₀)alkyl optionally substituted with one to three halo atoms, (C₁-C₁₀)alkoxy optionally substituted with one to three halo atoms, amino, hydroxy-(C₁-C₆)alkyl, (C₁-C₆)alkoxy-(C₁-C₆)alkyl, (C₁-C₆)-alkylamino, (C₁-C₆)alkyl-O—C(═O)—, (C₁-C₆)alkyl-O—C(═O)-(C₁-C₆)alkyl, (C₁-C₆)alkyl-C(═O)—O—, (C₁-C₆)alkyl-C(═O)-(C₁-C₆)alkyl-O, (C₁-C₆)alkyl-C(═O)—, (C₁-C₆)alkyl-C(═O)-(C₁-C₆)alkyl-di-(C₁-C₆)alkylamino, —C(═O)NH-(C₁-C₆)alkyl, (C₁-C₆)-alkyl-C(═O)—NH-(C₁-C₆)alkyl,

and pharmaceutically acceptable salts and solvates thereof.

In one embodiment of this practice, X¹ is hydrogen or (C₁-C₃)alkoxy optionally substituted with one to three fluorine atoms; X² and X³, which can be the same or different, are independently selected from the group consisting of hydrogen, (C₁-C₃)alkoxy optionally substituted with one to three fluorine atoms, and hydroxy; M is selected from the group consisting of —C(═O)—R³, —C(═O)—O—R³, and -(C₁-C₆)alkyl-NR⁵R ; R³ is H or (C₁-C₆) straight or branched alkyl; and R⁵ and R⁶, which can be the same or different, are each H or (C₁-C₆) straight or branched alkyl.

In another embodiment of this practice, X¹ is H or OCH₃; and X² and X³; which can be the same or different, are independently selected from the group consisting of hydrogen, —OCH₃, —OCF₃, and hydroxy.

In another practice, the invention relates to an isolated and purified compound having Formula IV: 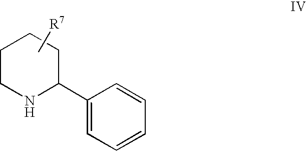
wherein R⁷ is amine or oxo;

and pharmaceutically acceptable salts and solvates thereof.

Preferably, the isolated and purified metabolites of the above Formulae II, III and IV are selected from the group consisting of:
2-[(2-phenyl-piperidine-3-ylamino)-methyl]-benzene-1,4-diol or a glucuronide congugate thereof,
2-aminomethyl-4-trifluoromethoxyphenol,
5-(2-methoxy-5-trifluoromethoxy-benzylamino)-6-phenyl-piperidin-2,4-dione,
2-aminomethyl-4-trifluoromethoxy-phenol or a sulfate conjugate or a glucuronide congugate thereof,
2-hydroxymethyl-4-trifluoromethoxy phenol or a glucuronide congugate thereof,
hydroxy 2-[(2-phenylpiperidine-3-ylamino)-methyl]-4-trifluoromethoxy-phenol or a glucuronide congugate thereof,
hydroxy 2-methoxy-5-trifluoromethoxy-benzyl-(2-phenyl-piperidin-3-yl)-amine or a glucuronide congugate thereof,
5-(2-hydroxy-5-trifluoromethoxy-benzylamino)-6-phenyl-piperidin-2-one or a glucuronide congugate thereof,
hydroxy 2-[(2-phenylpiperidin-3-ylamino)-methyl]-4-trifluoromethoxy-phenol or a glucuronide congugate thereof,
2-[(2-phenylpiperidin-3-ylamino)-methyl]-4-trifluoromethoxy-phenol or a glucuronide congugate thereof,
5-(2-hydroxy-5-trifluoromethoxy-benzylamino)-6-hydroxyphenol-piperidin-2-one or a glucuronide congugate thereof,
6-trifluoromethoxy-4H-benzo(1,3)oxazin-2-ol or a glucuronide congugate thereof,
hydroxy 2-[(2-phenylpiperidin-3-ylamino)-methyl]-4-trifluoromethoxy-phenol or a glucoronide conjugate thereof,
5-trifluoromethoxy salicyclic acid,
2-phenyl-3-amino-piperidine,
2-phenyl-3-oxo-piperidine,
2-methoxy-N-(2-phenyl-piperidin-3-yl)-5-trifluoromethoxy-benzamide,
2-[(2-phenylpiperidine-3-ylamino)-methyl]41trifluoromethoxy-phenol,
2-methoxy-5-trifluoromethoxy-benzyl-(2-phenyl-piperidin-3-yl)-amine and
5-(2-methoxy-5-trifluoromethoxy-benzylamino)-6-phenyl-piperidin-2-one.

The present invention also relates to the pharmaceutically acceptable acid addition and base salts of the metabolites of the compounds of Formula I, or analogues thereof. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., I,1′-methylene-bis-(2-hydroxy-3-naphthoate))salts.

The compounds of Formula I can have optical centers and thus occur in different enantiomeric configurations. The invention includes all enantiomers, diastereomers, and other stereoisomers and optical isomers of such compound of Formula I, as well as racemic and other mixtures thereof. For example, the compound of Formula I includes (R) and (S) enantiomers and cis and trans isomers. The present invention further includes all radiolabelled forms of the compound of formula I. Preferred radiolabelled compounds are those wherein the radiolabels are selected from as ³H, ¹³C, ¹⁴C, ¹⁸F, ¹²³I and ¹²⁵I. Such radiolabelled compounds are useful as research and diagnostic tools in metabolism pharmacokinetics studies, mass balances, quantitative and qualitative analysis of drug metabolites and in binding assays in animals and man.

In another practice, the invention relates to an assay for assessing the metabolic fate of (+)-(2S, 3S)-3-(2-methoxy-5-trifluoromethoxybenzylamino)-2-phenyl-piperidine, said assay comprising the metabolite of Formula (I) or an analogue thereof; preferably the metabolite of Formulae II, III or IV, individually or in any combination thereof.

In another practice, the invention relates to a pharmaceutical composition for antagonizing the effects of substance P in a mammal comprising a substance P antagonizing amount of an isolated and purified metabolite of a compound of Formula I, or an analogue thereof, preferably a compound of Formulae II, III or IV, or a pharmaceutically acceptable salt or solvate thereof, and a pharmaceutically acceptable carrier.

In another practice, the invention relates to a pharmaceutical composition for treating in a mammal a condition associated with the effect of excess substance P at its receptor site, comprising an amount of an isolated and purified metabolite of a compound of Formula I, or an analogue thereof, preferably a compound of Formulae II, III or IV, or a pharmaceutically acceptable salt or solvate thereof, effective in antagonizing the effect of substance P at its receptor site, and a pharmaceutically acceptable carrier.

In another practice, the invention relates to a pharmaceutical composition for treating in a mammal a condition associated with the effect of excess substance P at its receptor site, comprising an amount of an isolated and purified metabolite of a compound of Formula I, or an analogue thereof, preferably a compound of Formulae II, III or IV, or a pharmaceutically acceptable salt or solvate thereof, effective in treating said condition, and a pharmaceutically acceptable carrier.

In another practice, the invention relates to a pharmaceutical composition for treating in a mammal a condition selected from the group consisting of inflammatory diseases (e.g., arthritis, psoriasis, asthma or inflammatory bowel disease); anxiety; emesis; depressive disorders; colitis; psychosis; pain; gastroesophageal reflux disease; allergies such as eczema or rhinitis; chronic obstructive airways disease; hypersensitivity disorders such as poison ivy; vasospastic diseases such as angina, migraine and Reynaud's disease; fibrosing and collagen diseases such as scleroderma and eosinophilic fascioliasis; reflex sympathetic dystrophy such as shoulder/hand syndrome; addiction disorders such as alcoholism; stress related somatic disorders; peripheral neuropathy; neuralgia; neuropathological disorders such as Alzheimer's disease, AIDS related dementia, diabetic neuropathy or multiple sclerosis; disorders related to immune enhancement or suppression such as systemic lupus erythematosus; and rheumatic diseases such as fibrositis, preferably emesis and depressive disorders such as major depression, dysthymic disorders or Depressive Disorders Not Otherwise Specified, comprising an amount of an isolated and purified metabolite of a compound of Formula I, or an analogue thereof, preferably a compound of Formulae II, III or IV, or a pharmaceutically acceptable salt or solvate thereof, effective in antagonizing the effect of substance P at its receptor site, and a pharmaceutically acceptable carrier.

In another practice, the invention relates to a pharmaceutical composition for treating in a mammal a condition selected from the group consisting of inflammatory diseases (e.g., arthritis, psoriasis, asthma or inflammatory bowel disease); anxiety; emesis; depressive disorders; colitis; psychosis; pain; gastroesophageal reflux disease; allergies such as eczema or rhinitis; chronic obstructive airways disease; hypersensitivity disorders such as poison ivy; vasospastic diseases such as angina, migraine and Reynaud's disease; fibrosing and collagen diseases such as scleroderma and eosinophilic fascioliasis; reflex sympathetic dystrophy such as shoulder/hand syndrome; addiction disorders such as alcoholism; stress related somatic disorders; peripheral neuropathy; neuralgia; neuropathological disorders such as Alzheimer's disease, AIDS related dementia, diabetic neuropathy or multiple sclerosis; disorders related to immune enhancement or suppression such as systemic lupus erythematosus; and rheumatic diseases such as fibrositis; preferably emesis and depressive disorders such as major depression, dysthymic disorders or Depressive Disorders Not Otherwise Specified, comprising an amount of an isolated and purified metabolite of a compound of Formula I, or an analogue thereof, preferably a compound of Formulae II, III or IV, or a pharmaceutically acceptable salt or solvate thereof, effective in treating said condition, and a pharmaceutically acceptable carrier.

In another practice, the invention relates to a pharmaceutical composition for treating a condition in a mammal the treatment or prevention of which is effected or facilitated by a decrease in substance P mediated neurotransmission, comprising an amount of an isolated and purified metabolite of a compound of Formula I, or an analogue thereof, preferably a compound of Formulae II, III or IV, or a pharmaceutically acceptable salt or solvate thereof, effective in antagonizing the effect of substance P at its receptor site, and a pharmaceutically acceptable carrier.

In another practice, the invention relates to a pharmaceutical composition for treating a condition in a mammal the treatment or prevention of which is effected or facilitated by a decrease in substance P mediated neurotransmission, comprising an amount of an isolated and purified metabolite of a compound of Formula I, or an analogue thereof, preferably a compound of Formulae II, III or IV, or a pharmaceutically acceptable salt or solvate thereof, effective in treating said condition, and a pharmaceutically acceptable carrier.

In another practice, the invention relates to a method of antagonizing the effects of substance P in a mammal comprising administering to said mammal a substance P antagonizing amount of an isolated and purified metabolite of a compound of Formula I, or an analogue thereof, preferably a compound of Formulae II, III or IV, or a pharmaceutically acceptable salt or solvate thereof.

In another practice, the invention relates to a method of treating in a mammal a condition associated with the effect of excess substance P at its receptor site, comprising administering to said mammal an amount of an isolated and purified metabolite of a compound of Formula I, or an analogue thereof, preferably a compound of Formulae II, III or IV, or a pharmaceutically acceptable salt or solvate thereof, effective in antagonizing the effect of substance P at its receptor site, wherein said mammal is in need of said treatment.

In another practice, the invention relates to a method for treating in a mammal a condition associated with the effect of excess substance P at its receptor site, comprising administering to said mammal an amount of an isolated and purified metabolite of a compound of Formula I, or an analogue thereof, preferably a compound of Formulae II, III or IV, or a pharmaceutically acceptable salt or solvate thereof, effective in treating said condition, wherein said mammal is in need of said treatment.

In another practice, the invention relates to a method of treating in a mammal a condition selected from the group consisting of inflammatory diseases (e.g., arthritis, psoriasis, asthma or inflammatory bowel disease); anxiety; emesis; depressive disorders; colitis; psychosis; pain; gastroesophageal reflux disease; allergies such as eczema or rhinitis; chronic obstructive airways disease; hypersensitivity disorders such as poison ivy; vasospastic diseases such as angina, migraine and Reynaud's disease; fibrosing and collagen diseases such as scleroderma and eosinophilic fascioliasis; reflex sympathetic dystrophy such as shoulder/hand syndrome; addiction disorders such as alcoholism; stress related somatic disorders; peripheral neuropathy; neuralgia; neuropathological disorders such as Alzheimer's disease, AIDS related dementia, diabetic neuropathy or multiple sclerosis; disorders related to immune enhancement or suppression such as systemic lupus erythematosus; and rheumatic diseases such as fibrositis; preferably emesis and depressive disorders such as major depression, dysthymic disorders or Depressive Disorders Not Otherwise Specified, comprising administering to said mammal an amount an isolated and purified metabolite of a compound of Formula I, or an analogue thereof, preferably a compound of Formulae II, III or IV, or a pharmaceutically acceptable salt or solvate thereof, effective in antagonizing the effect of substance P at its receptor site, wherein said mammal is in need of said treatment.

In another practice, the invention relates to a method of treating in a mammal a condition selected from the group consisting of inflammatory diseases (e.g., arthritis, psoriasis, asthma or inflammatory bowel disease); anxiety; emesis; depressive disorders; colitis; psychosis; pain; gastroesophageal reflux disease; allergies such as eczema or rhinitis; chronic obstructive airways disease; hypersensitivity disorders such as poison ivy; vasospastic diseases such as angina, migraine and Reynaud's disease; fibrosing and collagen diseases such as scleroderma and eosinophilic fascioliasis; reflex sympathetic dystrophy such as shoulder/hand syndrome; addiction disorders such as alcoholism; stress related somatic disorders; peripheral neuropathy; neuralgia; neuropathological disorders such as Alzheimer's disease, AIDS related dementia, diabetic neuropathy or multiple sclerosis; disorders related to immune enhancement or suppression such as systemic lupus erythematosus; and rheumatic diseases such as fibrositis; preferably emesis and depressive disorders such as major depression, dysthymic disorders or Depressive Disorders Not Otherwise Specified, comprising administering to said mammal an amount an isolated and purified metabolite of a compound of Formula I, or an analogue thereof, preferably a compound of Formulae II, III or IV, or a pharmaceutically acceptable salt or solvate thereof, effective in treating said condition, wherein said mammal is in need of said treatment.

In another practice, the invention relates to a method of treating a condition in a mammal the treatment or prevention of which is effected or facilitated by a decrease in substance P mediated neurotransmission, comprising administering to said mammal an amount of an isolated and purified metabolite of a compound of Formula I, or an analogue thereof, preferably a compound of Formulae II, III or IV, or a pharmaceutically acceptable salt or solvate thereof, effective in antagonizing the effect of substance P at its receptor site, wherein said mammal is in need of said treatment.

In another practice, the invention relates to a method of treating a condition in a mammal the treatment or prevention of which is effected or facilitated by a decrease in substance P mediated neurotransmission, comprising administering to said mammal an amount of an isolated and purified metabolite of a compound of Formula I, or an analogue thereof, preferably a compound of Formulae II, III or IV, or a pharmaceutically acceptable salt or solvate thereof, effective in treating said condition, wherein said mammal is in need of said treatment.

The metabolites of Formula I, or analogues thereof, can advantageously be used in conjunction with one or more other therapeutic agents. It is to be understood that the present invention covers the use of a metabolite of general Formula I, or an analogue thereof, or a pharmacologically acceptable salt or solvate thereof in combination with other therapeutic agents.

The chemist of ordinary skill will recognize that certain compounds of this invention will contain one or more atoms which can be in a particular stereochemical, tautomeric, or geometric configuration, giving rise to stereoisomers, tautomers, regio and configurational isomers. All such isomers and mixtures thereof are included in this invention. Hydrates and solvates of the compounds of this invention are also included.

The subject invention also includes isotopically-labeled compounds, which are identical to those shown in Formulae I-XXIV, among other compounds encompassed by the invention, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively.

Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, can be preferred in some circumstances. Isotopically labeled compounds of of this invention and prodrugs thereof can generally be prepared by carrying out the procedures exemplified below or those known in the art. ¹⁴C-labeled compounds of the invention can be prepared by the methods outlined and exemplified in U.S. Pat. No. 5,552,412 by substituting a readily available isotopically labeled reagent for a non-isotopically labeled reagent.

The compounds of the invention, in their substantially pure form or in mixtures of known composition, can be used as analytical standards for in vitro or in vivo metabolism studies or as intermediates for the chemical synthesis or biosynthesis of new chemical entities. The compounds can be isolated as solids or in solutions.

In the methods of treatment of the present invention, a metabolite can be administered to a subject directly, such as in a tablet, or the metabolite can be administered by being produced in the subject's body through metabolism. For example, a metabolite of the present invention can be effectively administered to a subject to treat a disease or condition by administering to the subject an amount of (+)-(2S, 3S)-3-(2-methoxy-5-trifluoromethoxybenzylamino)-2-phenyl-piperidine, (one single 30 mg free base oral dose to humans) after which administration, the desired metabolite is formed in the subject's body through metabolism. Moreover, the administration route and dosage of (+)-(2S, 3S)-3-(2-methoxy-5-trifluoromethoxybenzylamino)-2-phenyl-piperidine can be varied, as desired, to obtain desired in vivo concentrations and rates of production of a metabolite.

The pharmaceutically acceptable acid addition salts of the metabolites of this invention can be formed of the compound itself, or of any of its esters, and include the pharmaceutically acceptable salts which are often used in pharmaceutical chemistry. For example, salts can be formed with inorganic or organic acids such as hydrochloric acid, hydrobromic acid, hydroiodic acid, sulfonic acids including such agents as naphthalenesulfonic, methanesulfonic and toluenesulfonic acids, sulfuric acid, nitric acid, phosphoric acid, tartaric acid, pyrosulfuric acid, metaphosphoric acid, succinic acid, formic acid, phthalic acid, lactic acid and the like, most preferable with hydrochloric acid, citric acid, benzoic acid, maleic acid, acetic acid and propionic acid.

The metabolites of this invention, as discussed above, can be administered in the form of pharmaceutically acceptable salts. The salts are conveniently formed, as is usual in organic chemistry, by reacting a metabolite of this invention, when basic, with a suitable acid, such as have been described above. The salts are quickly formed in high yields at moderate temperatures, and often are prepared by merely isolating the metabolite from a suitable acidic wash as the final step of the synthesis. The salt-forming acid is dissolved in an appropriate organic solvent, or aqueous organic solvent, such as an alkanol, ketone or ester. On the other hand, if a metabolite of this invention is desired in the free base form, it is isolated from a basic final wash step, according to the usual practice. A preferred technique for preparing hydrochlorides is to dissolve the free base in a suitable solvent and dry the solution thoroughly, as over molecular sieves, before bubbling hydrogen chloride gas through it.

When used as a medicament, the dose of a compound of this invention to be administered to a human is rather widely variable and subject to the judgement of the attending physician. It should be noted that it can be necessary to adjust the dose of a metabolite when it is administered in the form of a salt, such as a laureate, the salt forming moiety of which has an appreciable molecular weight. Effective administration can be range from about 5 mg/day-1500 mg/day, preferably about 30 mg/day. Of course, it is often practical to administer the daily dose of compound in portions, at various hours of the day. However, in any given case, the amount of compound administered will depend on such factors as the solubility of the active component, the formulation used and the route of administration.

The route of administration of the metabolites of this invention is not critical. The metabolites can be absorbed from the alimentary tract, however, the metabolites can be administered percutaneously, or as suppositories for absorption by the rectum, if desired in a given instance. All of the usual types of compositions can be used,. including tablets, chewable tablets, capsules, solutions, parenteral solutions, troches, suppositories and suspensions. Compositions are formulated to contain a daily dose, or a convenient fraction of daily dose, in a dosage unit, which can be a single tablet or capsule or convenient volume of a liquid.

In general, all of the compositions are prepared according to methods typically in pharmaceutical chemistry and/or isolated from in vivo or in vitro metabolism reactions such as those exemplified herein. The parent compound, (+)-(2S, 3S)-3-(2-methoxy-5-trifluoromethoxybenzylamino)-2-phenyl-piperidine, is prepared by those procedures outlined and/or exemplified in U.S. Pat. No. 5,773,450. The metabolites can be synthesized directly or can be formed by in vitro or in vivo enzymatic or metabolic reactions such as those described in the Examples.

Methods of formulation are well known in the art and are disclosed, for example, in Remington: The Science and Practice of Pharmacy, Mack Publishing Company, Easton, Pa., 19th Edition (1995). Pharmaceutical compositions for use within the present invention can be in the form of sterile, non-pyrogenic liquid solutions or suspensions, coated capsules, suppositories, lyophilized powders, transdermal patches or other forms known in the art.

Capsules are prepared by mixing the metabolite with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the metabolite. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant can used in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which facilitate the disintegration of a tablet to release a metabolite when the tablet becomes wet. They include starches, clays, celluloses, algins and gums, more particularly, corn and potato starches, methylcellulose, agar, bentonite, wood cellulose, powdered natural sponge, cation-exchange resins, alginic acid, guar gum, citrus pulp and carboxymethylcellulose, for example, can be used as well as sodium lauryl sulfate.

Tablets are often coated with sugar as a flavor and sealant, or with film-forming protecting agents to modify the dissolution properties of the tablet. The metabolites can also be formulated as chewable tablets, by using large amounts of pleasant-tasting substances such as mannitol in the formulation, as is now well-established in the art.

When the metabolites of the invention are administered as suppository, the typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the metabolites can be delayed or prolonged by proper formulation. For example, a slowly soluble pellet of the metabolite can be prepared and incorporated in a tablet or capsule. The technique can be improved by making pellets of several different dissolution rates and filling capsules with a mixture of the pellets. Tablets or capsules can be coated with a film which resists dissolution for a predictable period of time. Even the parenteral preparations can be made long-acting, by dissolving or suspending the metabolite in oily or emulsified vehicles which allow it to disperse only slowly in the serum.

The activity of the compounds of the present invention as substance P antagonists can be determined by their ability to inhibit the binding of substance P at its receptor sites in bovine caudate tissue, employing radioactive ligands to visualize the tachykinin receptors by means of autoradiography. The substance P antagonizing activity of the herein described compounds can be evaluated by using the standard assay procedure described by M. A. Cascieri et al., as reported in the Journal of Biological Chemistry, Vol. 258, p. 5158 (1983). This method essentially involves determining the concentration of the individual compound required to reduce by 50% the amount of radiolabelled substance P ligands at their receptor sites in said isolated cow tissues, thereby affording characteristic IC₅₀ values for each compound tested.

In this procedure, bovine caudate tissue is removed from a −70° C. freezer and homogenized in 50 volumes (w./v.) of an ice-cold 50 mM Tris (i.e., trimethamine which is 2-amino-2-hydroxymethyl-1,3-propanediol) hydrochloride buffer having a pH of 7.7. The homogenate is centrifuged at 30,000×G for a period of 20 minutes. The pellet is resuspended in 50 volumes of Tris buffer, rehomogenized and then recentrifuged at 30,000×G for another twenty-minute period. The pellet is then resuspended in 40 volumes of ice-cold 50 mM Tris buffer (pH 7.7) containing 2 mM of calcium chloride, 2 mM of magnesium chloride, 40 g/ml of bacitracin, 4 μg/ml of leupeptin, 2 μg of chymostatin and 200 g/ml of bovine serum albumin. This step completes the production of the tissue preparation.

The radioligand binding procedure is then carried out in the following manner, viz, by initiating the reaction via the addition of 100 μl of the test compound made up to a concentration of 1 μM, followed by the addition of 100 μl of radioactive ligand made up to a final concentration 0.5 mM and then finally by the addition of 800 μl of the tissue preparation produced as described above. The final volume is thus 1.0 ml, and the reaction mixture is next vortexed and incubated at room temperature (ca. 20° C) for a period of 20 minutes. The tubes are then filtered using a cell harvester, and the glass fiber filters (Whatman GF/B) are washed four times with 50 mM of Tris buffer (pH 7.7), with the filters having previously been presoaked for a period of two hours prior to the filtering procedure. Radioactivity is then determined in a Beta counter at 53% counting efficiency, and the IC₅₀ values are calculated by using standard statistical methods.

The anti-psychotic activity of the compounds of the present invention as neuroleptic agents for the control of various psychotic disorders is determined primarily by a study of their ability to suppress substance P-induced or substance P agonist induced hypermotility in guinea pigs. This study is carried out by first dosing the guinea pigs with a control compound or with an appropriate test compound of the present invention, then injecting the guinea pigs with substance P or a substance P agonist by intracerebral administration via canula and thereafter measuring their individual locomotor response to said stimulus.

The compositions of the present invention can be formulated in a conventional manner using one or more pharmaceutically acceptable carriers. Thus, the active compounds of the invention can be formulated for oral, buccal, intranasal, parenteral (e.g., intravenous, intramuscular or subcutaneous) or rectal administration or in a form suitable for administration by inhalation or insufflation.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition can take the form of tablets or lozenges formulated in a conventional manner.

The compounds of the invention can be formulated for parenteral administration by injection, including using conventional catheterization techniques or infusion. Formulations for injection can be presented in unit dosage form, e.g., in ampules or in multi-dose containers, with an added preservative. The compositions can take such forms as suspensions, solutions or emulsions in oily or aqueous vehicles, and can contain formulating agents such as suspending, stabilizing and/or dispersing agents. Alternatively, the active ingredient can be in powder form for reconstitution with a suitable vehicle, e.g., sterile pyrogen-free water, before use.

The compounds of the invention can also be formulated in rectal compositions such as suppositories or retention enemas, e.g., containing conventional suppository bases such as cocoa butter or other glycerides.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules and cartridges (made, for example, from gelatin) for use in an inhaler or insulator can be formulated containing a powder mix of a compound of the invention and a suitable powder base such as lactose or starch.

A proposed dose of the compound of the invention for oral, parenteral or buccal administration to the average adult human for the treatment of the conditions referred to above (e.g., depression) is 0.1 to 200 mg of the compound per unit dose which could be administered, for example, 1 to 4 times per day.

Aerosol formulations for treatment of the conditions referred to above (e.g., migraine) in the average adult human are preferably arranged so that each metered dose or “puff” of aerosol contains 20 μg to 1000 μg of the compound of the invention. The overall daily dose with an aerosol will be within the range 100 μg to 10 mg. Administration can be several times daily, for example 2, 3, 4 or 8 times, giving for example, 1, 2 or 3 doses each time.

It is to be noted that the compound of the invention can be administered either alone or in combination with pharmaceutically acceptable carriers by either of the routes previously indicated, and that such administration can be carried out in both single and multiple dosages. More particularly, the compound or combinations of compounds of the invention with other compounds can be administered in a wide variety of different dosage forms, i.e., they can be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of the invention are present in such dosage forms at concentration levels ranging from about 0.5% to about 90% by weight of the total composition.

A proposed daily dose of an active compound of this invention in the combination formulation for oral, parenteral, rectal or buccal administration to the average adult human for the treatment of the conditions referred to above is from about 0.01 mg to about 2000 mg, preferably from about 0.1 mg to about 200 mg of the compound of the invention per unit dose which could be administered, for example, 1 to 4 times per day.

Aerosol combination formulations for treatment of the conditions referred to above in the average adult human are preferably arranged so that each metered dose or “puff” of aerosol contains from about 0.01 μg to about 100 mg of the compound of this invention, preferably from about 1 μg to about 10 mg of such compound. Administration can be several times daily, for example 2, 3, 4 or 8 times, giving for example, 1, 2 or 3 doses each time.

All references and patents cited herein are incorporated by reference.

In the schemes and examples below, the following terms are intended to have the following, general meaning:
° C.: degrees Celsius
d; doublet (spectral)
EtOAc: ethyl acetate
mg: milligrams
Hz: hertz
J: coupling constant (in NMR)
L: liter(s)
mM or mmol: millimoles
MHz: megahertz
m/e mass to charge ratio (in mass spectrometry)
NMR: nuclear magnetic resonance
ppm: parts per million
rt or RT: room temperature
s: singlet (NMR),
t: triplet (NMR)
μg: micrograms

The following schemes and examples are offered in illustration of the present invention; they are not to constrain the scope of the same in any way.

### EXAMPLE 1

### Isolation of (+)-(2S, 3S)-3-(2-methoxy-5-trifluoromethoxybenzylamino)-2-phenyl-piperidine

Materials and Methods

Six healthy male human subjects (4 extensive metabolizers (EM) of CYP2D6 and 2 poor metabolizers (PM) of CYP2D6) were administered in a single 30 mg (free base) oral dose of (+)-(2S, 3S)-3-(2-methoxy-5-trifluoromethoxybenzylamino)-2-phenyl-piperidine containing 100 *Ci of [14C]-(+)-(2S, 3S)-3-(2-methoxy-5-trifluoromethoxybenzylamino)-2-phenyl-piperidine (Lot # 49800-59-3, specific activity 1.22 mCi/mmol, radio purity of >99%). Blood samples were collected into red-top tubes (no preservatives or anticoagulant or serum separator) at the following time points: 1, 4, 8, 12, and 24 hours post dose. The blood samples were centrifuged at 4° C. and serum was transferred to clean tubes.

Extraction of Metabolites from Serum Samples

a) Serum samples were pooled by human subject separately (3 ml from each sampling time, total of 15 ml) and aliquots of 1.5 mL from each pool were extracted with 5 mL of acetonitrile. The mixtures were vortex mixed for 5 minutes and centrifuged at 3500 rpm for 5 minutes to remove the precipitated proteins. Supernatants were combined and a small aliquot of supernatant was counted. Approximately 96% (average of all subjects) of the radioactivity was recovered in the supernatant. The supernatants were evaporated under N² in a turbovap at room temperature. The residues were reconstituted with mobile phase and aliquots of 100 L for each subject were injected on the HPLC system for profiling and metabolite identification.

b). Urine samples were pooled according to sample volume. Approximately 2-5 mL of urine samples were evaporated under N₂ at room temperature in a Turbovap and reconstituted in 200 ul of 10 mM ammonium acetate (pH 4.0)/methanol/dimethyl sulfoxide (1.5:1.5:1). Aliquots of 100 μL were injected inot the HPLC-Arc system without further purification for profiling. Approximately 80 mL urine pools were dried in the Turbovap at room temperature and then reconstituted in 600 μL of 10 mM ammonium acetate (pH 4.0)/methanol/dimethyl sulfoxide (1.5:1.5:1). Aliquots of 80 μL were injected onto the HPLC-MS/MS system for metabolite identification.

c). Fecal samples were pooled according to sample weight. For metabolite profiling, approximately 1 g of each sample pool was extracted with 7 mL of acetonitrile/H₂O (6:1 v/v) twice, and 100 μL aliquots of both extractions were counted to determine recovery. Approximately 88.2% of the radioactivity was recovered in the supernatants. The supernatant was evaporated under N₂ in a Turbovap at room temperature and the residue was reconstituted at 500 μL of 10 mM ammonium acetate (pH 5.0)/methanol/dimethyl sulfoxide (1.5:1.5:1). Aliquots of 100 μL were injected onto the HPLC-Arc system for profiling. For metabolite identification, 10 g of each sample pool was extracted with 30 mL of Acetonitrile/H₂O (5:1 v/v) twice. The supernatants were combined and dried in Turbovap at room temperature. The dried residue was dissolved in 5 mL of water and extracted with 15 mL hexanes twice. The hexanes layer was removed and the aqueous layer was further extracted with 15 mL ethyl acetate twice. The extractions were combined and dried in the turbovap at room temperature and the residue was reconstituted in 360 μL of 10 mM ammonium acetate (pH 5.0)/methanol/dimethyl sulfoxide (1.5:1.5:1). Aliquots of 90 μL were injected onto HPLC-MS/MS system for metabolite identification.

Profiling and Quantitative Assessment of Metabolites in Serum

Measuring the radioactivity in the individual peaks separated on the HPLC column using a -RAM detector allowed the quantification of each metabolite. The -RAM provided a printout containing integrated regions of interest in cpm, the percentage of the radiolabeled material in these regions of interest, and radiochromatograms. The -RAM was operated in a homogeneous liquid scintillation counting mode with addition of 4 mL/min of Tru-Count scintillation cocktail to the HPLC column effluent post MS detection.

Qualitative assessment of the formation of 5-trifluoromethoxy salicylic acid in human liver S9 extract

Human liver S9 fraction (HL-1123) was used to examine the formation of 5-trifluoromethoxy salicylic acid. A solution of [14C] (+)-(2S, 3S)-3-(2-methoxy-5-trifluoromethoxybenzylamino)-2-phenyl-piperidine (˜27 M) was incubated with HL-1123 (37 mg/ml S9 protein) in the presence of 100 mM potassium phosphate buffer, pH 7.4, and cofactor solution (9 mM MgCl2, 0.54 mM NADP, 6.2 mM DL-Isocitric Acid, and 0.5 U/ml Isocitric Dehydrogenase) in a total volume of 10 ml. The reaction mixture was initiated by the addition of the S9 extract and incubation was carried out in a shaking water bath at 37° C. The reaction mixture was monitored at 1, 15 and 24 hr and stopped by the addition of ACN (5 mL). The mixture was vortex mixed for 5 minutes and centrifuged at 3500 rpm for 5 minutes to remove the precipitated proteins. The supernatant was evaporated under N² in a turbovap at room temperature. The residue was reconstituted with mobile phase and aliquot of 100 L for each reaction was injected on the HPLC system for profiling.

5-trifluoromethoxy salicylic acid metabolite was isolated and purified from 15 and 24 hr hepatic S9 incubation mixtures using the 10 mM NH₄OAc (pH 5)/ACN HPLC system described below. The purified HPLC fraction (27-30 min) was dried under N² stream and stored at −20° C. until use.

Using the methods described above, the following compounds were isolated:
glucuronide congugate of 2-[(2-phenyl-piperidine-3-ylamino)-methyl]-benzene-1,4-diol m/z 479
2-aminomethyl-4-trifluoromethoxyphenol m/z 208
5-(2-methoxy-5-trifluoromethoxy-benzylamino)-6-phenyl-piperidin-2,4-dione m/z 409
sulfate conjugate of 2-aminomethyl-4-trifluoromethoxy-phenol m/z 305
glucuronide congugate of 2-hydroxymethyl-4-trifluoromethoxy phenol m/z 385
glucuronide congugate of hydroxy 2-[(2-phenylpiperidine-3-ylamino)-methyl]-4-trifluoromethoxy-phenol m/z 735
glucuronide congugate of hydroxy 2-methoxy-5-trifluoromethoxy-benzyl-(2-phenyl-piperidin-3-yl)-amine 573
glucuronide congugate of 5-(2-hydroxy-5-trifluoromethoxy-benzylamino)-6-phenyl-piperidin-2-one m/z 557
glucuronide congugate of hydroxy 2-[(2-phenylpiperidin-3-ylamino)-methyl]-4-trifluoromethoxy-phenol m/z 559
glucuronide congugate of 2-[(2-phenylpiperidin-3-ylamino)-methyl]-4-trifluoromethoxy-phenol m/z 543
glucuronide congugate of 5-(2-hydroxy-5-trifluoromethoxy-benzylamino)-6-hydroxyphenol-piperidin-2-one m/z 410
glucuronide congugate of 6-trifluoromethoxy-4H-benzo(1,3)oxazin-2-ol m/z 395
glucoronide conjugate of hydroxy 2-[(2-phenylpiperidin-3-ylamino)-methyl]-4-trifluoromethoxy-phenol m/z 559
5-trifluoromethoxy salicyclic acid m/z 221
2-phenyl-3-amino-piperidine m/z 177
2-phenyl-3-oxo-piperidine m/z 176
2-methoxy-N-(2-phenyl-piperidin-3-yl)-5-trifluoromethoxy-benzamide m/z 397
hydroxy 2-[(2-phenylpiperidin-3-ylamino)-methyl]-4-trifluoromethoxy-phenol m/z 383
2-[(2-phenylpiperidine-3-ylamino)-methyl]-41trifluoromethoxy-phenol m/z 367
2-methoxy-5-trifluoromethoxy-benzyl-(2-phenyl-piperidin-3-yl)-amine m/z 381
5-(2-methoxy-5-trifluoromethoxy-benzylamino)-6-phenyl-piperidin-2-one m/z 381
5-(2-methoxy-5-trifluoromethoxy-benzylamino)-6-phenyl-piperidin-2-one m/z 395

### EXAMPLE 2

### Chemical Synthesis of 5-trifluoromethoxy Salicylic Acid

A. Synthesis of 2-Methoxy-5-trifluoromethoxy-benzoic acid

To a solution of 2-bromo-1-methoxy4-trifluoromethoxy-benzene (1.0 g, 3.69 mmol) in anhydrous ether (50 mL), at −78° C., under a nitrogen atmosphere, was added n-BuLi (0.738 mL, 1.85 mmol, 2.5M solution in hexane) over 5 minutes, while magnetically stirring. After 15 minutes, anhydrous carbon dioxide was bubbled through the reaction mixture for 10 minutes. It was allowed to warm up to room temperature. The ethereal solution was washed with a 5% solution of aqueous sodium hydroxide (3×50 mL). The combined aqueous solution was acidified with 1N solution of hydrochloric acid (pH 2) then extracted with Ether (3×200 mL). The combined ethereal solution was dried over anhydrous sodium sulfate and concentrated to give ˜0.26 g of 2-methoxy-5-trifluoromethoxy-benzoic acid.

B. Synthesis of 2-Hydroxy-5-trifluoromethoxy-benzoic acid (5-trifluoromethoxy salicylic acid)

To a solution of 2-methoxy-5-trifluoromethoxy-benzoic acid (23.6 mg, 0.1 mmol) in anhydrous methylene chloride (0.9 mL) was added, boron tribromide (0.100 mL, 1M solution in methylene chloride) at 0° C. The reaction was allowed to continue for 16 hours. The solution was made acidic (pH=0) using 1N solution of hydrochloric acid, and extracted with ethyl acetate (3×3 ml). The ethyl acetate solution was dried over anhydrous sodium sulfate, filtered and concentrated to give ˜11 mg of 2-hydroxy-5-trifluoromethoxy-benzoic acid (5-trifluoromethoxy salicylic acid.

Nuclear Magnetic Resonance

Nuclear magnetic resonance experiments were performed at 400 MHz (1H) Typical 1D proton experiments were performed over the spectral range 0-8 or 0-13 parts per million (ppm).

Results

5-trifluoromethoxy Salicylic Acid

5-trifluoromethoxy salicylic acid had a retention time of approximately 28.5 minutes on the 10 mM ammonium acetate (pH 5.0)/ACN HPLC system and had poor ionization efficiency. It was detected in all EM and PM subjects and accounted for approximately 56% and 29% of the total radioactivity for EM and PM subjects, respectively.

Total ion current (TIC) response of the negative ion ESI mass spectrum of the HPLC purified fraction showed a deprotonated molecular ion [M-H]- at m/z 221 (FIG. 1). The CID mass spectrum of 5-trifluoromethoxy salicylic acid had a deprotonated molecular ion at m/z 221 suggesting 5-trifluoromethoxy salicylic acid was a cleaved product with zero or an even number or nitrogen atoms.

2-Methoxy-5-trifluoromethoxy-benzoic Acid

Resonances were observed at 13.03 ppm (broad s, 1H) corresponding to —COOH, 3.81 ppm (s, 3H) corresponding to —OCH3, 7.55-7.49 (m, 2H) and 7.2 ppm (d, 1H, J=8.7 Hz) consistent with a ring system containing three substitutions, and at 2.47 ppm (m) and 3.32 ppm (broad s) corresponding to DMSO (partially deuterated) and H₂O, respectively. Based on the above data, the carboxylation product of 2-bromo-1-methoxy-4-trifluoromethoxy-benzene was identified as 2-methoxy-5-trifluoromethoxy-benzoic acid.

5-trifluoromethoxy Salicylic Acid

Resonances were observed at 3.81 ppm (s, 3H). Corresponding to —OCH3 (impurity from the starting material 2-methoxy-5-trifluoromethoxy-benzoic acid), 2.47 ppm (m) and 1.95 ppm (s) corresponding to DMSO (partially deuterated) and ETOAc, respectively, and at 7.64 ppm (d, 1H, J=2.9 Hz), 7.53-7.50 ppm (dd, 1H, J=9.1, 2.9 Hz), and 7.05 ppm (d, 1H, J=9.1 Hz) which are consistent with a ring system containing three substitutions.

Based on a reading of the present description and claims, certain modifications to the compositions and methods described herein will be apparent to one of ordinary skill in the art. The claims appended hereto are intended to encompass these modifications.

1. An isolated and purified metabolite of the compound of Formula (I): 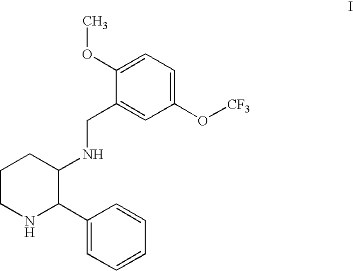
or an analogue thereof, or a pharmaceutically acceptable salt or solvate thereof.

2. An isolated and purified compound having Formula (II): 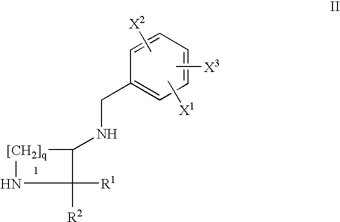
wherein X¹, X² and X³, which can be the same or different, are each independently selected from O-Gluc, —C(═O)OH, OH, —OSO₂OH, (C₁-C₁₀)alkoxy and (C₁-C₁₀)alkyl, wherein said (C₁-C₁₀)alkoxy and (C₁-C₁₀)alkyl are each optionally substituted with one to three halo atoms;
R¹ is a radical selected from the group consisting of H, (C₁-C₆) straight or branched alkyl, (C₃-C₇)cycloalkyl, (C₃-C₇)heterocycloalkyl, aryl, benzhydryl, and O-Gluc, wherein one of the phenyl moieties of said benzhydryl can optionally be replaced by naphthyl, and phenyl(C₁-C₆)alkyl-, wherein said aryl group and the phenyl moieties of said phenyl(C₁-C₆)alkyl- and benzhydryl can optionally be substituted with one or more substituents independently selected from halo, nitro, O-gluc, (C₁-C₁₀)alkyl optionally substituted with one to three halo atoms, (C₁-C₁₀)alkoxy optionally substituted with one to three halo atoms, amino, hydroxy-(C₁-C₆)alkyl, (C₁-C₆)alkoxy-(C₁-C₆)alkyl, (C₁-C₆)-alkylamino, (C₁-C₆)alkyl-O—C(═O)—, (C₁-C₆)alkyl-O—C(═O)-(C₁-C₆)alkyl, (C₁-C₆)alkyl-C(═O)—O—, (C₁-C₆)alkyl-C(═O)-(C₁-C₆)alkyl-O, (C₁-C₆)alkyl-C(═O)—, (C₁-C₆)alkyl-C(═O)-(C₁-C₆)alkyl-di-(C₁-C₆)alkylamino, —C(═O)NH-(C₁-C₆)alkyl, (C₁-C₆)-alkyl-C(═O)—NH-(C₁-C₆)alkyl, —NHC(═O)H and —NHC(═O)-(C₁-C₆)alkyl; and;
R² is hydrogen, phenyl or (C₁-C₆)alkyl; or
R¹ and R², together with the carbon to which they are attached, form a saturated carbocyclic ring having 3 to 7 carbon atoms wherein one of said carbon atoms can optionally be replaced by oxygen, nitrogen or sulfur;
q is an integer from 1-5;
ring 1 can be substituted with 1 or 2 oxo groups;
or a pharmaceutically acceptable salt or solvate thereof.
with the provisos that when X¹ is —OCH₃, X² is not —OCF₃, and when X² is —OCH₃, X¹ is not —OCF₃.

3. The isolated and purified compound according to claim 2, wherein
X¹ and X², which can be the same or different, are each (C₁-C₃)alkoxy optionally substituted with one to three fluorine atoms;
R¹ is aryl;
R is H; and
q is 2.

4. The isolated and purified compound according to claim 3, wherein
X¹ and X², which can be the same or different, are each —OCF₃,
R¹ is phenyl,
R is H, and
q is 2.

5. An isolated and purified compound having Formula (III): 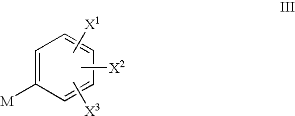
wherein X¹, X² and X³, which can be the same or different, are independently selected from the group consisting of halo, hydrogen, nitro, O-Gluc, (C₁-C₁₀)alkyl optionally substituted with one to three halo atoms, (C₁-C₁₀)alkoxy optionally substituted with one to three halo atoms,
—O—SO₂—OH, trifluoromethyl, hydroxy, phenyl, cyano, amino, (C₁-C₆)-alkylamino, di-(C₁-C₆)alkylamino, —C(═O)—NH-(C₁-C₆)alkyl, (C₁-C₆)alkyl-C(═O)—NH-(C₁-C₆)alkyl, hydroxy(C₁-C₄)alkyl, (C₁-C₄)alkoxy(C₁-C₄)alkyl, —NHC(═O)H and —NHC(═O)-(C₁-C₆)alkyl;
M is selected from the group consisting of —C(═O)—R³, —C(═O)—O—R³, -(C₁-C₆)alkyl-C(═O)—R³, -(C₁-C₆)alkyl-C(═O)—OR⁴, and -(C₁-C₆)alkyl-NR⁵R⁶;
R³ and R⁴, which can be the same or different, are each independently selected from the group consisting of H, (C₁-C₆) straight or branched alkyl, (C₃-C₇)cycloalkyl, (C₃-C₇)heterocycloalkyl, aryl, (C₁-C₆)aryl, benzhydryl wherein one of the phenyl moieties of said benzhydryl can optionally be replaced by naphthyl, and wherein said aryl group and aryl moiety of said aryl(C₁-C₆)alkyl- and benzhydryl can optionally be substituted with one or more substituents independently selected from the group consisting of halo, nitro, (C₁-C₁₀)alkyl optionally substituted with one to three halo atoms, (C₁-C₁₀)alkoxy optionally substituted with one to three halo atoms, amino, hydroxy-(C₁-C₆)alkyl, (C₁-C₆)alkoxy-(C₁-C₆)alkyl, (C₁-C₆)-alkylamino, (C₁-C₆)alkyl-O—C(═O)—, (C₁-C₆)alkyl-O—C(═O)-(C₁-C₆)alkyl, (C₁-C₆)alkyl-C(═O)—O—, (C₁-C₆)alkyl-C(═O)-(C₁-C₆)alkyl-O, (C₁-C₆)alkyl-C(═O)—, (C₁-C₆)alkyl-C(═O)-(C₁-C₆)alkyl-di-(C₁-C₆)alkylamino, —C(═O)N H-(C₁-C₆)alkyl, (C₁-C₆)-alkyl-C(═O)—NH-(C₁-C₆)alkyl, —NHC(═O)H and —NHC(═O)-(C₁-C₆)alkyl;
R⁵ and R⁶, which can be the same or different, are each independently selected from the group consisting of H, (C₁-C₆) straight or branched alkyl, (C₃-C₇)cycloalkyl, (C₃-C₇)heterocycloalkyl, aryl, (C₁-C₆)aryl, benzhydryl wherein one of the phenyl moieties of said benzhydryl can optionally be replaced by naphthyl, and wherein said aryl group and aryl moiety of said aryl(C₁-C₆)alkyl- and benzhydryl can optionally be substituted with one or more substituents independently selected from the group consisting of halo, nitro, (C₁-C₁₀)alkyl optionally substituted with one to three halo atoms, (C₁-C₁₀)alkoxy optionally substituted with one to three halo atoms, amino, hydroxy-(C₁-C₆)alkyl, (C₁-C₆)alkoxy-(C₁-C₆)alkyl, (C₁-C₆)-alkylamino, (C₁-C₆)alkyl-O—C(═O)—, (C₁-C₆)alkyl-O—C(═O)-(C₁-C₆)alkyl, (C₁-C₆)alkyl-C(═O)—O—, (C₁-C₆)alkyl-C(═O)-(C₁-C₆)alkyl-O, (C₁-C₆)alkyl-C(═O)—, (C₁-C₆)alkyl-C(═O)-(C₁-C₆)alkyl-di-(C₁-C₆)alkylamino, —C(═O)NH-(C₁-C₆)alkyl, (C₁-C₆)-alkyl-C(═O)—NH-(C₁-C₆)alkyl,
or a pharmaceutically acceptable salt or solvate thereof.

6. The isolated and purified compound according to claim 5, wherein X¹ is hydrogen or (C₁-C₃)alkoxy optionally substituted with one to three fluorine atoms;
X² and X³, which can be the same or different, are independently selected from the group consisting of hydrogen, (C₁-C₃)alkoxy optionally substituted with one to three fluorine atoms, and hydroxy;
M is selected from the group consisting of —C(═O)—R³, —C(═O)—O—R³, and -(C₁-C₆)alkyl-NR⁵R⁶;
R³ is H or (C₁-C₆) straight or branched alkyl; and
R⁵ and R⁶, which can be the same or different, are each H or (C₁-C₆) straight or branched alkyl.

7. The isolated and purified compound according to claim 6, wherein
X¹ is H or OCH₃; and
X² and X³, which can be the same or different, are each independently selected from the group consisting of hydrogen, —OCH₃, —OCF₃, and hydroxy.

8. An isolated and purified compound having Formula IV: 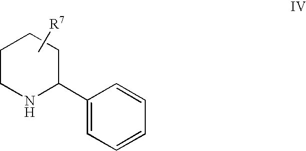
wherein R⁷ is amine or oxo;
or a pharmaceutically acceptable salt or solvate thereof, racemic-diastereomeric mixtures or optical isomers thereof, or prodrugs thereof.

9. The isolated and purified metabolite of claim 1 selected from the group consisting of:
2-[(2-phenyl-piperidine-3-ylamino)-methyl]-benzene-1,4-diol or a glucuronide congugate thereof,
2-aminomethyl-4-trifluoromethoxyphenol,
5-(2-methoxy-5-trifluoromethoxy-benzylamino)-6-phenyl-piperidin-2,4-dione,
2-aminomethyl-4-trifluoromethoxy-phenol or a sulfate conjugate or a glucuronide congugate thereof,
2-hydroxymethyl-4-trifluoromethoxy phenol or a glucuronide congugate thereof,
hydroxy 2-[(2-phenylpiperidine-3-ylamino)-methyl]-4-trifluoromethoxy-phenol or a glucuronide congugate thereof,
hydroxy 2-methoxy-5-trifluoromethoxy-benzyl-(2-phenyl-piperidin-3-yl)-amine or a glucuronide congugate thereof,
5-(2-hydroxy-5-trifluoromethoxy-benzylamino)-6-phenyl-piperidin-2-one or a glucuronide congugate thereof,
hydroxy 2-[(2-phenylpiperidin-3-ylamino)-methyl]-4-trifluoromethoxy-phenol or a glucuronide congugate thereof,
2-[(2-phenylpiperidin-3-ylamino)-methyl]-4-trifluoromethoxy-phenol or a glucuronide congugate thereof,
5-(2-hydroxy-5-trifluoromethoxy-benzylamino)-6-hydroxyphenol-piperidin-2-one or a glucuronide congugate thereof,
6-trifluoromethoxy-4H-benzo(1,3)oxazin-2-ol or a glucuronide congugate thereof,
hydroxy 2-[(2-phenylpiperidin-3-ylamino)-methyl]-4-trifluoromethoxy-phenol or a glucoronide conjugate thereof,
5-trifluoromethoxy salicyclic acid,
2-phenyl-3-amino-piperidine,
2-phenyl-3-oxo-piperidine,
2-methoxy-N-(2-phenyl-piperidin-3-yl)-5-trifluoromethoxy-benzamide,
2-[(2-phenylpiperidine-3-ylamino)-methyl]41trifluoromethoxy-phenol,
2-methoxy-5-trifluoromethoxy-benzyl-(2-phenyl-piperidin-3-yl)-amine and
5-(2-methoxy-5-trifluoromethoxy-benzylamino)-6-phenyl-piperidin-2-one.

10. An assay for assessing the metabolic fate of (+)-(2S, 3S)-3-(2-methoxy-5-trifluoromethoxybenzylamino)-2-phenyl-piperidine comprising the isolated and purified metabolite of claim 1.

11. A pharmaceutical composition for antagonizing the effects of substance P in a mammal, comprising a substance P antagonizing amount of an isolated and purified metabolite of a compound according to claim 1, or an analogue thereof, or a pharmaceutically acceptable salt or solvate thereof, and a pharmaceutically acceptable carrier.

12. A pharmaceutical composition for treating in a mammal a condition associated with the effect of excess substance P at its receptor site, comprising an amount of an isolated and purified metabolite of a compound according to claim 1, or an analogue thereof, or a pharmaceutically acceptable salt or solvate thereof, effective in antagonizing the effect of substance P at its receptor site, and a pharmaceutically acceptable carrier.

13. A pharmaceutical composition for treating in a mammal a condition associated with the effect of excess substance P at its receptor site, comprising an amount of an isolated and purified metabolite of a compound according to claim 1, or an analogue thereof, or a pharmaceutically acceptable salt or solvate thereof, effective in treating said condition and a pharmaceutically acceptable carrier.

14. A pharmaceutical composition for treating in a mammal a condition selected from the group consisting of inflammatory diseases, anxiety, emesis, depressive disorders, colitis, psychosis, pain, gastroesophageal reflux disease, allergies, chronic obstructive airways disease, hypersensitivity disorders, vasospastic diseases, fibrosing and collagen diseases, reflex sympathetic dystrophy, addiction disorders, stress related somatic disorders, peripheral neuropathy, neuralgia, neuropathological disorders, disorders related to immune enhancement or suppression, and rheumatic diseases, comprising an amount of an isolated and purified metabolite of a compound according to claim 1, or an analogue thereof, or a pharmaceutically acceptable salt or solvate thereof, effective in antagonizing the effect of substance P at its receptor site, and a pharmaceutically acceptable carrier.

15. A pharmaceutical composition for treating in a mammal a condition selected from the group consisting of inflammatory diseases, anxiety, emesis, depressive disorders, colitis, psychosis, pain, gastroesophageal reflux disease, allergies, chronic obstructive airways disease, hypersensitivity disorders, vasospastic diseases, fibrosing and collagen diseases, reflex sympathetic dystrophy, addiction disorders, stress related somatic disorders, peripheral neuropathy, neuralgia, neuropathological disorders, disorders related to immune enhancement or suppression, and rheumatic diseases, comprising an amount of an isolated and purified metabolite of a compound according to claim 1, or an analogue thereof, or a pharmaceutically acceptable salt or solvate thereof, effective in treating said condition, and a pharmaceutically acceptable carrier.

16. A pharmaceutical composition for treating a condition in a mammal, the treatment or prevention of which is effected or facilitated by a decrease in substance P mediated neurotransmission, comprising an amount of an isolated and purified metabolite of a compound according to claim 1, or an analogue thereof, or a pharmaceutically acceptable salt or solvate thereof, effective in antagonizing the effect of substance P at its receptor site, and a pharmaceutically acceptable carrier.

17. A pharmaceutical composition for treating a condition in a mammal, the treatment or prevention of which is effected or facilitated by a decrease in substance P mediated neurotransmission, comprising an amount of an isolated and purified metabolite of a compound according to claim 1, or an analogue thereof, or a pharmaceutically acceptable salt or solvate thereof, effective in treating said condition, and a pharmaceutically acceptable carrier.

18. A method of antagonizing the effects of substance P in a mammal comprising administering to said mammal a substance P antagonizing amount of an isolated and purified metabolite of a compound according to claim 1, or an analogue thereof, or a pharmaceutically acceptable salt or solvate thereof.

19. A method of treating in a mammal a condition associated with the effect of excess substance P at its receptor site, comprising administering to said mammal an amount of an isolated and purified metabolite of a compound according to claim 1, or an analogue thereof, or a pharmaceutically acceptable salt or solvate thereof, effective in antagonizing the effect of substance P at its receptor site, wherein said mammal is in need of said treatment.

20. A method of treating in a mammal a condition associated with the effect of excess substance P at its receptor site, comprising administering to said mammal an amount of an isolated and purified metabolite of a compound according to claim 1, or an analogue thereof, or a pharmaceutically acceptable salt or solvate thereof, effective in treating said condition, wherein said mammal is in need of said treatment.

21. A method of treating in a mammal a disease condition selected from the group consisting of inflammatory diseases, anxiety, emesis, depressive disorders, colitis, psychosis, pain, gastroesophageal reflux disease, allergies, chronic obstructive airways disease, hypersensitivity disorders, vasospastic diseases, fibrosing and collagen diseases, reflex sympathetic dystrophy, addiction disorders, stress related somatic disorders, peripheral neuropathy, neuralgia, neuropathological disorders, disorders related to immune enhancement or suppression, and rheumatic diseases, comprising administering to said mammal an amount an isolated and purified metabolite of a compound according to claim 1, or an analogue thereof, or a pharmaceutically acceptable salt or solvate thereof, effective in antagonizing the effect of substance P at its receptor site, wherein said mammal is in need of said treatment.

22. A method of treating in a mammal a condition selected from the group consisting of inflammatory diseases, anxiety, emesis, depressive dissorders, colitis, psychosis, pain, gastroesophageal reflux disease, allergies, chronic obstructive airways disease, hypersensitivity disorders, vasospastic diseases, fibrosing and collagen diseases, reflex sympathetic dystrophy, addiction disorders, stress related somatic disorders, peripheral neuropathy, neuralgia, neuropathological disorders, disorders related to immune enhancement or suppression, and rheumatic diseases, comprising administering to said mammal an amount of an isolated and purified metabolite of a compound according to claim 1, or an analogue thereof, or a pharmaceutically acceptable salt or solvate thereof, effective in treating said condition, wherein said mammal is in need of said treatment.

23. A method of treating a condition in a mammal, the treatment or prevention of which is effected or facilitated by a decrease in substance P mediated neurotransmission, comprising administering to said mammal an amount of an isolated and purified metabolite of a compound according to claim 1, or an analogue thereof, or a pharmaceutically acceptable salt or solvate thereof, effective in antagonizing the effect of substance P at its receptor site, wherein said mammal is in need of said treatment.

24. A method of treating a condition in a mammal, the treatment or prevention of which is effected or facilitated by a decrease in substance P mediated neurotransmission, comprising administering to said mammal an amount of an isolated and purified metabolite of a compound according to claim 1, or an analogue thereof, or a pharmaceutically acceptable salt or solvate thereof, effective in treating said condition, wherein said mammal is in need of said treatment.

25. The pharmaceutical composition according to claim 14, wherein said condition or disorder is emesis or a depressive disorder selected from major depression, a dysthymic disorder or Depressive Disorders Not Otherwise Specified.

26. The pharmaceutical composition according to claim 15, wherein said condition or disorder is emesis or a depressive disorder selected from major depression, a dysthymic disorder or Depressive Disorders Not Otherwise Specified.

27. The method according to claim 21, wherein said condition or disorder is emesis or a depressive disorder selected from major depression, a dysthymic disorder or Depressive Disorders Not Otherwise Specified.

28. The method according to claim 22, wherein said condition or disorder is emesis or a depressive disorder selected from major depression, a dysthymic disorder or Depressive Disorders Not Otherwise Specified.
